(19) United States
(12) Patent Application Publication
(10) Pub. No.: US 2025/0001183 A1
(43) Pub. Date: Jan. 2, 2025

(54) SYSTEM AND METHOD FOR SIMULTANEOUSLY MODULATING ASCENDING AND DESCENDING NEURAL PAIN PATHWAYS USING SPINAL CORD STIMULATION

(71) Applicant: Wavegate Corporation, Lake Charles, LA (US)

(72) Inventor: Erich W. Wolf, II, Lake Charles, LA (US)

(73) Assignee: Wavegate Corporation, Lake Charles, LA (US)

(21) Appl. No.: 18/886,674

(22) Filed: Sep. 16, 2024

Related U.S. Application Data
(63) Continuation-in-part of application No. 17/443,172, filed on Jul. 21, 2021, now Pat. No. 12,090,322.
(60) Provisional application No. 62/705,891, filed on Jul. 21, 2020.

(51) Int. Cl. A61N 1/36; A61N 1/02; A61N 1/05

(52) U.S. Cl. CPC A61N 1/36189; A61N 1/025; A61N 1/0551; A61N 1/36062; A61N 1/36071; A61N 1/36171; A61N 1/36175

(57) ABSTRACT

The invention relates to a system and method for improving spinal cord stimulation (SCS) by simultaneously modulating ascending and descending pain pathways. The invention utilizes a Randomized Intermittent Dichotomous Stimulation (RIDS) paradigm, introducing a randomly timed target stimulation signal interspersed with a background stimulation signal. This novel method enhances the cognitive processing of pain signals by utilizing event-related potentials, such as the P300 brain potential, to activate both inhibitory pathways and minimize pain habituation. The target stimulus, distinct from the background SCS signal, elicits a cognitive response by prompting the patient to recognize the target and perform a physical or mental task. This simultaneous modulation of pain pathways increases treatment efficacy, extends the longevity of SCS therapy, and reduces habituation effects, offering an optimized therapeutic solution for chronic pain management.

### CROSS-REFERENCE TO RELATED APPLICATIONS

This application is a continuation-in-part of U.S. application Ser. No. 17/443,172 filed on Jul. 21, 2021, now U.S. Pat. No. 12,090,322 granted on Sep. 17, 2024, which claims priority benefits from U.S. Provisional Application No. 62/705,891 filed on Jul. 21, 2020. The patent applications identified above are incorporated herein by reference in their entirety to provide continuity of disclosure.

### FIELD OF THE INVENTION

The field of the invention is randomized intermittent stimulation for enhancing long-term efficacy of spinal cord stimulation.

### BACKGROUND OF THE INVENTION

Nociceptive pain results from activation of mechanoreceptive nerve fibers within the musculoskeletal system which project to pain pathways within the central nervous system. Nociceptive pain is typically protective, being elicited in response to potentially tissue-damaging forces within the musculoskeletal system. Upon cessation of the offending biomechanical forces, the nociceptive fibers typically cease firing and the pain resolves. However, tissue damage can result in these nociceptive fibers erroneously remaining activated chronically, despite the resolution of the initial traumatic response, giving rise to chronic nociceptive pain.

Neuropathic pain is an alternate form of pain arising from direct nerve injury. Other forms of chronic pain, such as Complex Regional Pain Syndrome (CRPS), may be mediated through imbalance of the autonomic nervous system. Functional ability may be severely impacted by chronic pain, which often is refractory to pharmacological or surgical treatment.

In most cases of chronic pain, spinal cord stimulation (“SCS”) can be an effective treatment by modulating physiological transmission of pain signals from the periphery to the brain, the so called “ascending pain pathways.” SCS treatment requires that electrical impulses be applied to the spinal cord via an electrode array implanted within the spinal canal.

In most cases of chronic nociceptive pain, spinal cord stimulation (“SCS”) can be an effective treatment by modulating physiological transmission of pain signals from the periphery to the brain, the so called “ascending pain pathways.” SCS treatment requires that electrical impulses be applied to the spinal cord via an electrode array implanted adjacent the spinal canal.

Referring to FIG. 1, SCS typically requires an implanted pulse generator (IPG) 32 which delivers electrical impulses to the spinal cord through the electrode lead 31 connected to an electrode array 30. A separate controller 33 is in communication with IPG 32 and transmits operational instructions to it. The IPG is typically contained in a titanium canister which is implanted subcutaneously and draws power from a battery. One of skill will understand that the pulse generator may also be an external pulse generator (EPG) and the leads may be percutaneous leads, as is often used for initial efficacy testing before IPG implantation. Examples are shown in U.S. Pat. No. 11,890,466 to Wolf II, incorporated herein by reference for all purposes.

Referring to FIG. 2, detail of electrode array 30 is shown. Electrode array 30 typically includes electrode contacts 35 sealed into elastomeric housing 36 and is known as a surgical lead. One of skill will understand that stimulation may also be delivered by percutaneous leads, such as shown and described in U.S. Pat. No. 11,890,466 to Wolf II. In either case, each electrode contact has a separate electrical conductor in electrode lead 31 so that the current to each contact may be independently controlled. Independent control allows the applied electric field across the spinal cord surface to be spatially adjusted.

Referring also to FIGS. 3 and 4, placement of the electrode array will be further described. Spinal column 1 has a number of vertebrae, categorized as lumbar vertebrae 2, thoracic vertebrae 3, cervical vertebrae 4 and sacral vertebrae 5. Representative vertebra 10, a thoracic vertebra, has a number of notable features which are in general shared with lumbar vertebrae 2 and cervical vertebrae 4. The thick oval segment of bone forming the anterior aspect of vertebra 10 is vertebral body 12. Vertebral body 12 is attached to bony vertebral arch 13 through which spinal nerves 11 run. Vertebral arch 13, forming the posterior of vertebra 10, is comprised of two pedicles 14a that extend from the sides of vertebral body 12 and laminae 15. Superior articular facets 14b project from pedicles 14a and join in a triangle to form a hollow archway, spinal canal 16. Surrounding spinal cord 20 is dura 21 that contains cerebrospinal fluid (CSF) 22. Epidural space 24 is the space within the spinal canal lying outside the dura. Electrode array 30 is typically positioned in epidural space 24 between dura 21 and the walls of spinal canal 16 towards the dorsal aspect of the spinal canal nearest laminae 15 and spinous process 17.

In use, the IPG delivers a stimulation signal consisting of pulses of electrical current to the electrode array, which travel through the electrodes to targeted neurons within the ascending tracts of the spinal cord. The resulting electric field disrupts the perception of pain by depolarizing the targeted neurons. Applying the correct amplitude for the stimulation signal is important. Applying inadequate current will fail to depolarize the targeted neurons, rendering the treatment ineffective, while applying excess current will stimulate excess cell populations which results in a noxious sensation. Charge balancing of the stimulation signal is also important. Signals that are not charge balanced risk tissue damage.

SCS has been employed for treatment of pain since the early 1970's and is particularly suitable for treating chronic pain since it does not require precise targeting of the anatomical pain generator, nor is it limited to a specific nociceptive, neuropathic, or autonomic pathway. This makes SCS treatment widely flexible to adapt to different patient conditions. SCS also offers minimally invasive procedure for implantation, reduced opioid use and overall improved quality of life.

The reason that SCS treatment of pain is effective is not well understood. The prevailing theory is the “durable gate theory” proposed by Ronald Melzack and Patrick Wall in 1965. Durable gate theory suggests that pain perception by the brain is controlled by activation of inhibitory interneurons which downregulate firing of projection neurons within the ascending pain pathways of the spinal cord. Theoretically, SCS achieves reduction of pain by activation of inhibitory interneurons within the ascending pain pathways.

Melzack also concluded that pain is a multidimensional phenomenon, having numerous sensory, affective, cognitive, and evaluative components, heavily implicating the role of the brain in overall pain perception. The emotional and cognitive components of pain are well recognized and their importance is clinically evidenced by the success of cognitive behavioral therapy (“CBT”). CBT relies on training the patient to change their behavior by identifying and changing negative thought patterns. CBT has been important in treating chronic pain by improving the patient's understanding of their condition and teaching pain management skills.

Anatomically, the emotional and cognitive components of pain are processed within the medial prefrontal cortex (“MPC”) which is composed of the anterior cingulate cortex (“ACC”), prelimbic cortex, and infralimbic cortex. Functional magnetic resonance imaging (“fMRI”) studies indicate that there is increased activation of the ACC and decreased activity in the limbic cortices due to chronic pain. The ascending pain pathways from the spinal cord project to the medial thalamus, relays nociceptive pain information to the insula, ACC, and somatosensory cortices for “discriminative” processing by the brain. Discriminative processing allows the patient to identify the intensity, location, and memory of the pain. Likewise, the MPC, insula, and limbic system are associated with the affective component of pain and pain memory. Efferent pathways from the MPC project to the periaqueductal grey (“PAG”) which exerts descending inhibitory influence on spinal pain pathways. The interaction of the ACC, MPC, and PAG during pain processing are thought to create a “descending inhibitory pathway” that inhibits the perception of pain by the brain.

An example of the descending inhibitory pathway may be found in the cognitive process of placebo analgesia. Placebo analgesia occurs when the administration of placebos, which are substances that lack active ingredients, leads to pain relief. The pain relief is considered a result of the patient's belief that an analgesic drug or other medical intervention is being received. Placebo analgesia has been shown to be effective for controlling the perception of pain, stress-related insomnia, and to manage cancer treatment side effects like fatigue and nausea. The exact mechanisms of placebo analgesia are still not fully understood but are believed to require a cognitive process that increases the level of neurotransmitters, like endorphins and dopamine, in certain brain regions linked to mood, emotional reaction, and self-awareness.

Cognitive neuroscience uses event related potential (“ERP”) studies to examine brain activity related to sensory, cognitive, or motor events. Such studies have shown that randomly intermittent stimuli, or so called “odd-ball” or “target” stimulus, embedded within a tonic pattern of visual, auditory, or somatosensory “background” stimuli will elicit event related brain potentials, such as the N100, P200 and P300 brain potentials, when the subject is asked to recognize the target stimulus. Resulting event-related potentials have been shown to be dependent upon factors such as the probability of the target stimulus (how often it occurs), the intensity of the target stimulus, and the level of difference between the target stimulus and the background stimuli.

Magnetoencephalography has demonstrated that use of a random target stimulus will elicit event-related potentials localized to the inferior parietal lobule/supramarginal gyrus, with subsequent activation of the anterior cingulate cortex and dorsolateral prefrontal cortex.

Similarly, anticipation of a painful stimulus can induce a prolonged event-related brain potential known as the contingent negative variation (“CNV”), suggesting this to be a correlate of the cognitive component of pain memory. The CNV is a slow cortical endogenous potential that is widely recognized as the electrophysiological signature of a task-specific preparatory state that facilitates stimulus perception and the required response. It was first described by Gary Walter in 1964 as an “expectancy wave.” CNV has been useful as a biomarker for neurocognitive disorders, such as Alzheimer's disease. The CNV further indicates the existence and potential usefulness of cognitive processes in inhibiting pain in the descending inhibitory pathway.

Despite the many benefits of SCS, it has not been effectively combined with cognitive studies of pain or event related potentials. SCS works at the spinal cord level to affect the “gate” of the pain but its influence on the cognitive and evaluative components of pain is poorly understood. Further, SCS treatment still exhibits several potential problems or complications. For example, SCS treatment alone can exhibit decreased effectiveness over time. Decreased effectiveness over time is known as “habituation” and occurs for reasons not well understood. Thus, there remains a need for the combination of cognitive studies with SCS to increase the effectiveness of SCS treatment, and for reducing or eliminating habituation.

### SUMMARY OF THE INVENTION

The inventor has recognized that chronic intermittent exacerbations of pain have many physiological and psychological similarities to the random target stimulus used to elicit event-related potentials in cognitive studies, but with the additional activation of the insular and limbic systems mediating the affective pain component and pain memory.

The inventor has also recognized that a target SCS stimulation signal, randomly occurring in a typical background SCS stimulation signal, provided by spinal cord stimulator, used in conjunction with cognitive therapy involving a task upon recognition of the target signal, inhibits both the ascending pain pathways and the descending pain pathways simultaneously, thereby increasing the effectiveness of the SCS stimulation signal and reducing habituation.

More specifically, the inventor has recognized that known SCS signals may be used to activate the descending pain-inhibitory pathway without eliciting activation of the affective and memory components of the somatosensory pathways. In traditional modulation of the ascending pain pathways within the spinal cord via SCS, a continuous background electrical signal is applied to the dorsal aspect of the spinal cord, which may or may not be perceptible to the patient. The background electrical signal is generally applied to the region of the spinal cord which somatotopically corresponds to the painful area. But, by introducing a perceptible change to the background electrical signal using a so called “target” signal, which is perceptibly different to the subject from the background electrical signal, and then simultaneously tasking the subject to take some action when the target signal is perceived, event-related potentials (such as the P300) will result. The event related potentials then activate the dorsolateral prefrontal cortex and ACC, with efferents to the periaqueductal grey, but avoid activation of the prelimbic and infralimbic cortex.

Aside from modulating the descending pain-inhibitory pathways, a non-noxious somatotopically matched target SCS signal also leads to reduction in pain memory, analogous to the tactile attention-shift training and peak end rule memory work components of cognitive behavioral therapy.

The Randomized Intermittent Dichotomous Stimulation (“RIDS”) signal disclosed provides a target spinal cord stimulation signal randomly inserted into a background spinal cord stimulation signal, thus providing a somatotopically-matched non-noxious stimulus to replace the cognitive attention to the painful stimulus. During therapy, the target stimulation signal is then intentionally paired with a physical or mental task for the subject to perform when the target stimulation signal is recognized, thereby reinforcing selective attention to the target stimulation signal.

By combining a conventional spinal cord stimulation signal with a randomly injected target stimulation signal, while simultaneously modulating the cognitive evaluation of pain, pain can be reduced in both the ascending and descending inhibitory pathways, thus improving SCS efficacy and longevity and reducing habituation.

The RIDS signal consists of a background SCS signal which is randomly interrupted by a “target” stimulus signal. While several background SCS signals are preferred, the background SCS signal may be any signal that modulates the electrophysiological function of the spinal cord to reduce the perception of pain.

The background stimulation signal may be any sequence of stimuli for which there is inhibitory modulation of ascending pain pathways at the spinal cord. The target signal is a novel stimulus that is injected into the background signal at random intervals. The target signal is preferably somatotopically matched to the painful region. A physical or implicit (virtual) task is then assigned to the patient to accomplish, when the target signal is recognized, to potentiate the cognitive response to the selective attention. The incidence of the target signal should not be so sparse as to lose selective attention nor so frequent that it becomes non-novel.

### BRIEF DESCRIPTION OF THE DRAWINGS

In the detailed description of the preferred embodiments presented below, reference is made to the accompanying drawings.

FIG. 1 is a lateral view of the human spine showing the approximate position of an electrode array for spinal cord stimulation of the prior art.

FIG. 2 shows a prior art surgical electrode array with leads for spinal cord stimulation of the prior art.

FIG. 3 shows an axial view of a thoracic vertebra indicating the position of the spinal cord and an electrode array for spinal cord stimulation of the prior art.

FIG. 4 shows a sagittal view of the human spine showing the approximate position of an electrode array in the dorsal epidural space for spinal cord stimulation of the prior art.

FIG. 5 shows a schematic of an IPG and transcutaneous control system of a preferred embodiment.

FIG. 6A is an architecture diagram of a preferred embodiment of an IPG.

FIG. 6B is an architecture diagram of a preferred RIDS circuit.

FIG. 6C is a schematic of a preferred N−1 comparator.

FIG. 6D is a schematic of a preferred linear feedback register circuit.

FIG. 6E is an architecture diagram of a preferred embodiment of a transcutaneous control system.

FIG. 6F is an architecture diagram of a preferred embodiment of an administration device.

FIG. 7A is a diagram of a preferred RIDS signal.

FIG. 7B shows a preferred background burst stimulation waveform.

FIG. 7C shows a preferred background tonic stimulation waveform.

FIG. 7D shows a preferred background high frequency stimulation waveform.

FIG. 7E is a diagram of a preferred background waveform signal.

FIG. 7F is a diagram of a preferred target waveform signal, paired to the preferred background waveform signal.

FIGS. 8A and 8B are flow charts of a preferred embodiment of an IPG program implementing a preferred RIDS paradigm.

FIGS. 8C and 8D is a flow chart of a preferred embodiment of an IPG program implementing a preferred RIDS signal.

FIG. 9 is a state chart for operation of a preferred control system.

FIG. 10 shows a patient set-up for a preferred RIDS therapy session.

FIG. 11 is a flowchart of a preferred treatment method for a patient implementing a RIDS therapy session with an attention monitoring task.

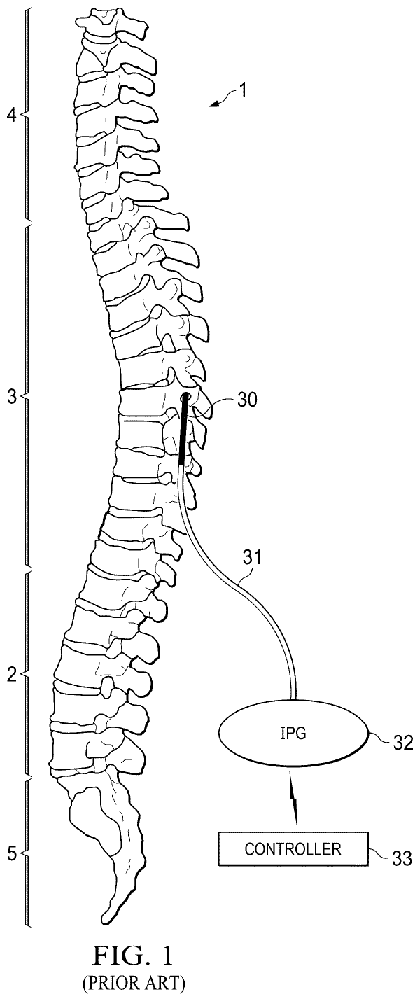

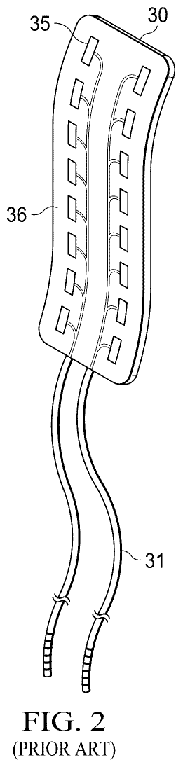

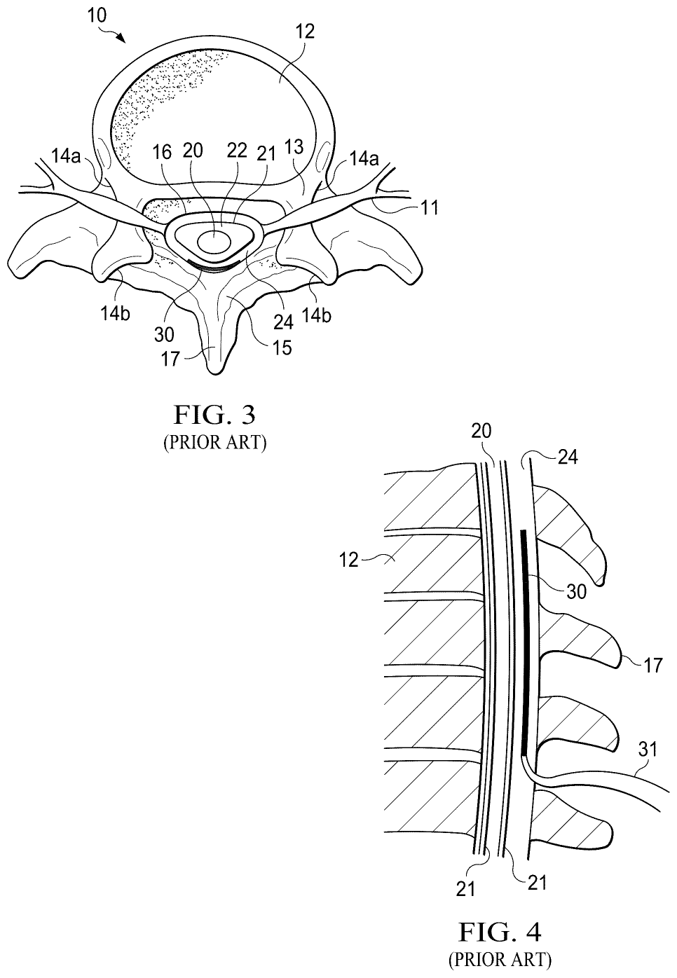

### DETAILED DESCRIPTION OF THE INVENTION

In the description that follows, like parts are marked throughout the specification and figures with the same numerals, respectively. The figures are not necessarily drawn to scale and may be shown in exaggerated or generalized form in the interest of clarity and conciseness. Unless otherwise specified, all uses of the terms “about” and “approximately” refer to a tolerance of ±20%.

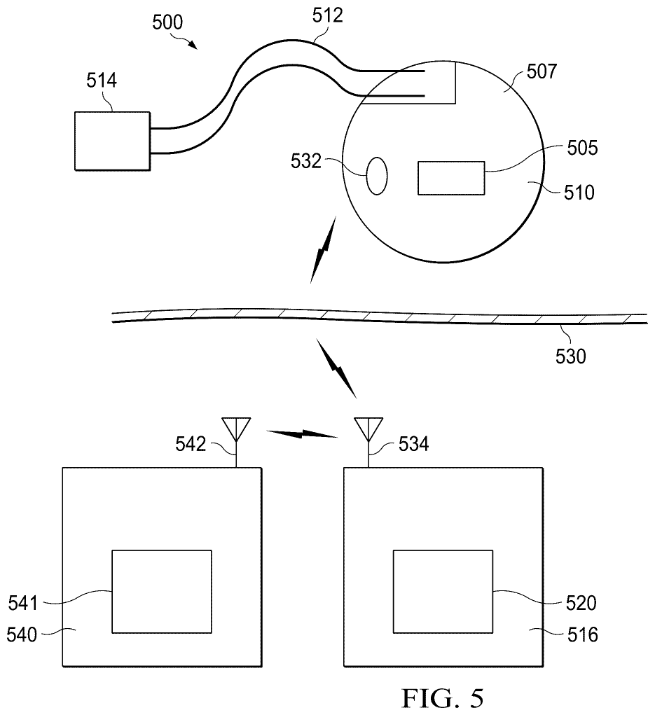

Referring then to FIG. 5, preferred IPG system 500 comprises an IPG 510 implanted subcutaneously beneath skin surface 530.

IPG 510 comprises controller 505, operatively connected to RF antenna 532, as will be further described. IPG 510 is preferably battery operated and contained by hermetically sealed case 507, which provides for long term subcutaneous implantation. Surgical lead 514, is implanted in the epidural space of the spinal canal, as previously described.

In use, controller 505 receives signals from RF antenna 532, for use in communicating data and receiving instructions for operation of the IPG, as will be further described. When active, the controller sends modulated electrical pulses through electrode leads 512 to surgical lead 514, as will be further described.

The IPG system further comprises external system manager 516. External system manager 516 includes controller 520, operatively connected to RF antenna 534.

In use, controller 520 includes a set of instructions which generates a set of operational parameters which are sent to the IPG wirelessly from RF antenna 534 to RF antenna 532, as will be further described.

The IPG system further comprises administrator device 540. Administrator device 540 includes controller 541, operatively connected to RF antenna 542.

In use, controller 541 includes a set of instructions which causes the upload of a data wirelessly from RF antenna 542, containing data and button press indications received from external system manager 516, as will be further described.

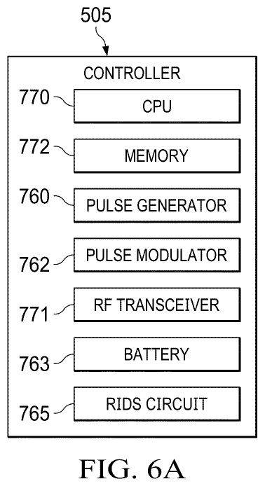

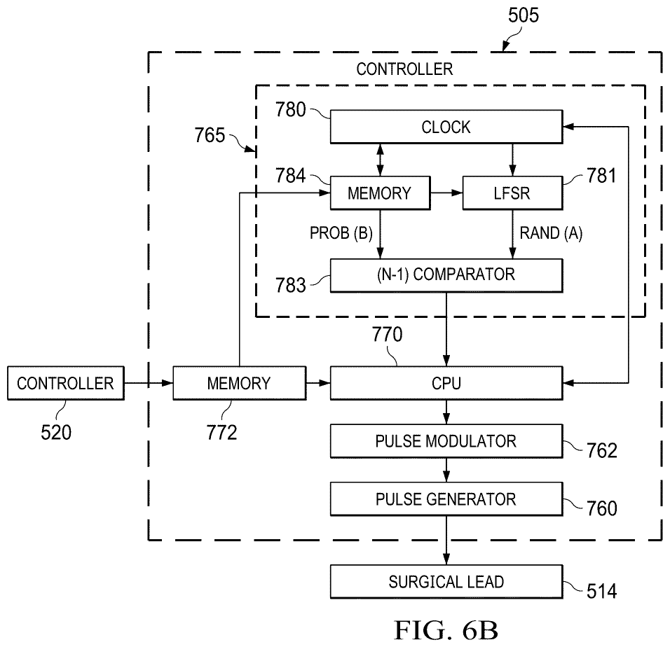

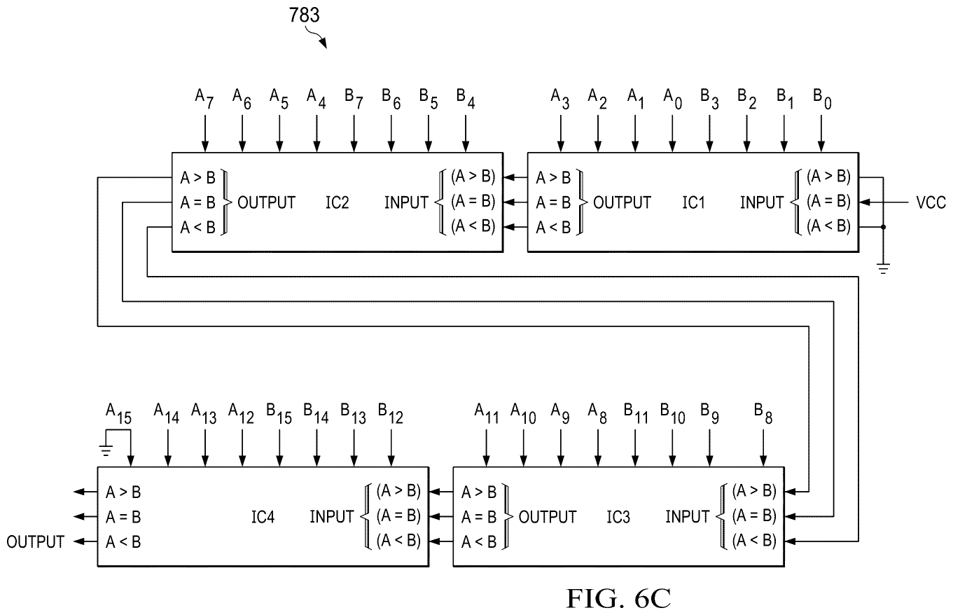

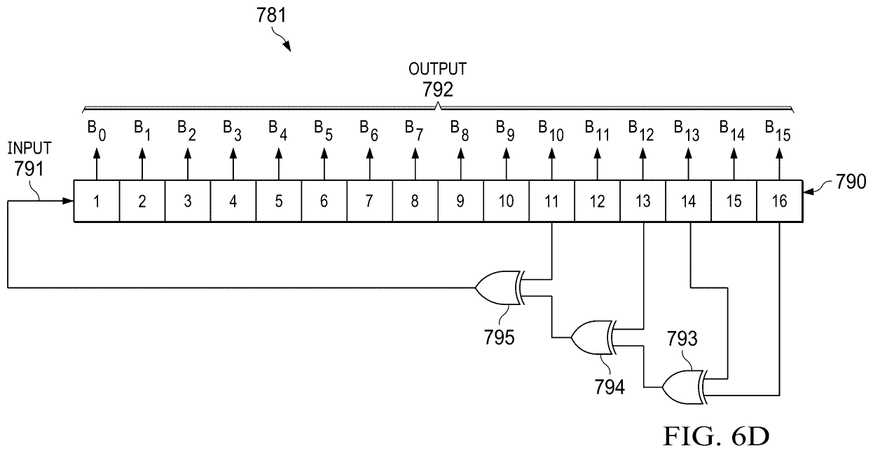

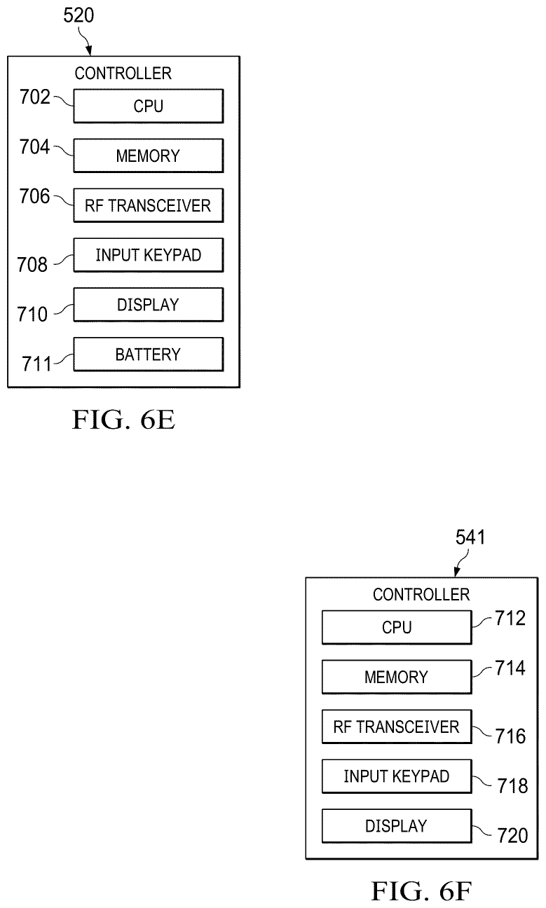

Referring to FIG. 6A, controller 505 further comprises CPU 770 including onboard memory 772. Controller 505 is further operatively connected to RF transceiver 771, for transmission of status signals and reception of control signals. CPU 770 is further operatively connected to RIDS circuit 765, pulse modulator 762 and pulse generator 760 for generation and transmission of stimulation signals. All components are operatively connected to battery 763, which provides current to operate controller 505.

In order to generate a stimulation pulse to the electrodes, the CPU consults onboard memory 772 to determine pulse width, pulse frequency and pulse amplitudes for each electrode contact. The parameters are transmitted to pulse modulator 762 which modulates the stimulation waveform signal based on the input parameters. The waveform signal is passed to pulse generator 760. Pulse generator 760 then provides the requisite current to each of the electrode contacts within the surgical lead.

Referring then to FIG. 6B, RIDS circuit 765 will be further described. RIDS circuit 765 is generally a timer circuit and serves in an embodiment as an epoch timer. RIDS circuit 765 further comprises clock 780. Clock 780 is set to a clock frequency relative to the epoch length, as will be further described. Clock 780 is operatively connected to linear feedback shift register 781, memory 784 and CPU 770. Memory 784 is operatively connected to (or incorporated with) memory 772 and receives binary instructions from that memory, as will be further described. Memory 784 is also operatively connected to N−1 comparator 783. Likewise, linear feedback shift register 781 is operatively connected to N−1 comparator 783. The output of N−1 comparator 783 is connected to CPU 770.

Preferably, clock 780 is implemented within CPU 770 such as the MSP430 family from Texas Instruments of Dallas, Texas. Preferably, RF transceiver 771 is part no. ST25RV3993-HPEV, available from STMicorelectronics N.V. of Plan-les-Ouates, Switzerland.

Referring then to FIG. 6C, N−1 comparator 783 will be further described.

N−1 comparator 783 is preferably comprised of four, 4-bit comparator ICs, IC1, IC2, IC3 and IC4. The A>B and A<B inputs of IC1 are tied to ground. The A=B input of IC1 is tied to VCC. The A>B, A=B and A<B outputs of IC1 are connected to the A>B, A=B and A<B inputs of IC2, respectively. The A>B, A=B, and A<B, outputs of IC2 are tied to the A>B, A=B and A<B inputs of IC3, respectively. The A>B, A=B, and A<B outputs of IC3 are tied to the A>B, A=B and A<B inputs of IC4, respectively. The A<B output of IC4 is connected to CPU 770, as previously described. The A>B and A=B outputs of IC4 are allowed to float.

The B0 through B15 inputs of IC1, IC2, IC3 and IC4 are connected to memory 784 and receive a binary number, called the “probability number,” as will be further described. Likewise, inputs A0 through A14 are connected to the output of linear feedback shift register 781, and receive the least significant 15-bits of the pseudo-random number generated by the linear feedback register, as will be further described. Input A15 is tied to ground, as will be further described.

Since the most significant bit of the “A” input to the N−1 comparator, A15, is set to ground, the comparator only compares the least significant 15-bits of the “A” input to the 16-bit “B” input and so is designated an “N−1 comparator.” In other embodiments, where the number of bits in the comparator inputs is other than 16-bits, the comparator is always structured to compare “N−1” bits of the “A” input to “N” bits of the “B” input, where N is the number of bits in the “B” input. As examples, the number of bits “N” in other embodiments may be 4, 8, 32 and 64 bits.

Preferably, N−1 comparator 783 is comprised of four cascaded 4-bit comparators, each part number SN74LS85, available from Texas Instruments.

Referring to FIG. 6D, linear feedback shift register 781 of a preferred embodiment will be further described.

Linear feedback shift register 781 preferably comprises a 16-bit shift register 790, having input 791 and output 792. Linear feedback shift register 781 is driven by three XOR gates 793, 794, and 795. XOR gate 793 taps the register at bit 16 and bit 14. The output of XOR gate 793 is operatively connected to a first input of XOR gate 794. Linear feedback shift register 781 is tapped at bit 13 to provide a second input to XOR gate 794. The output of XOR gate 794 forms a first input of XOR gate 795. Linear feedback shift register 781 is tapped at bit 11 for a second input to XOR gate 795. The output of XOR gate 795 forms input 791 to linear feedback shift register 781. On each clock pulse, the bits in the register are shifted one position to the right.

In other embodiments, the tap locations on the register may be different, as may the number of bits in the register itself. Likewise, one of skill will recognize that in other embodiments, XNOR gates, AND gates, OR gates, NAND gates, and NOR gates may be employed in place of the XOR gates.

The pseudo-random number “A” is accessed from all 16-bits of the shift register, output 792. Once the register is initialized with a seed value, it produces a different 16-bit pseudo-random number at the output every clock cycle.

In this preferred embodiment, a seed value is provided automatically by memory 784 to begin operation of the register. The seed value is read into the register when the circuit is initialized. The output of the register is deterministic and produces a string of repeating binary numbers in a set cycle. However, the cycle is very long. For example, this linear feedback register cycles through a maximum number of 65535 states, excluding the all 0 state. Other configurations of linear feedback registers may of course be used. The linear feedback register is also preferably implemented in CMOS, which requires extremely low power for operation. This is important because low power operation preserves battery life of the IPG.

Referring also again to FIG. 6C, when the RIDS circuit is in use, memory 784 provides the initial 16-bit seed to linear feedback shift register 781. The pseudo-random number which is output by linear feedback shift register 781, is sent to N−1 comparator 783.

A target probability number is input into memory 784 when the circuit is initialized from controller 520, in 16-bit form. The 16-bits of the target probability number “B” are clocked into N−1 comparator 783 and remain there unaltered during operation of the circuit. Because input A15 of the N−1 comparator is tied to ground, for each clock cycle, only the fifteen least significant bits of the pseudo random number are compared to the 16-bits of the target probability number. By eliminating the most significant bit of the binary pseudo-random number, the maximum number which it can be is reduced to half of its original value. Hence, the target probability number automatically has twice the number of potential values of the 15-bit pseudo-random number, and so has twice the probability of being greater than the 15-bit pseudo-random number. Since the target stimulation signal is delivered only when the target probability number is greater than the pseudo-random number, the maximum probability of the target stimulus being delivered is limited by hardware to 50%.

If the fifteen least significant bits of the pseudo-random number, “A,” from linear feedback shift register result is less than the value of the 16-bits of target probability number, “B,” resident in the digital comparator, then the output “A<B” is set high by the N−1 comparator. When the A<B output is high, an edge triggered interrupt is asserted and sent to CPU 770 by the N−1 comparator. Upon receipt of the edge triggered interrupt, the CPU is programmed to stop the background stimulus signal and start the target stimulus signal, for one clock cycle. At the next clock cycle, the CPU is programmed to stop the target stimulation signal and to resume the background stimulation signal, and to wait for the next edge triggered interrupt from the N−1 comparator. The parameters that define the target waveform and the parameters that define the background waveform are both stored in memory 772 and are accessed by CPU 770 as required.

In other hardware embodiments, the number of, “N−1,” comparator bits and the number of linear feedback shift register bits, “N,” may be chosen to provide at least one target stimulus occurrence approximately every day, at the epoch length, as will be described in further examples.

Referring to FIG. 6E, controller 520 will be further described. Controller 520 includes CPU 702 connected to RF transceiver 706, display 710, input keypad 708, and memory 704. In the preferred embodiment, display 710 is a low power liquid crystal display adapted to show the current operational state of the system. Input keypad 708 is a simple push button contact array, such as a 12 button digital keypad, which is constantly monitored by CPU 702. Memory 704 is onboard memory connected to CPU 702. In the preferred embodiment, RF transceiver 706 is a low power transmitter/receiver combination. In the preferred embodiment, all components of the controller draw power from onboard battery 711.

Referring to FIG. 6F, controller 541 will be further described. Controller 541 includes CPU 712, operatively connected to input keypad 718 RF transceiver 716, display 720, and memory 714. In the preferred embodiment, display 720 is a low power liquid crystal display adapted to show the current operational state of the system. Input keypad 718 is a simple push button contact array, such as a 12 button digital keypad, which is constantly monitored by CPU 712. Memory 714 is onboard memory connected to CPU 712. In the preferred embodiment, RF transceiver 716 is a low power transmitter/receiver combination.

In various preferred embodiments, the components of controller 541 and controller 520 may be included in a personal computer, such as a laptop or smart phone which transmits and receives RF signals containing data and instructions via Wi-Fi, infrared, Bluetooth, or other wireless protocols.

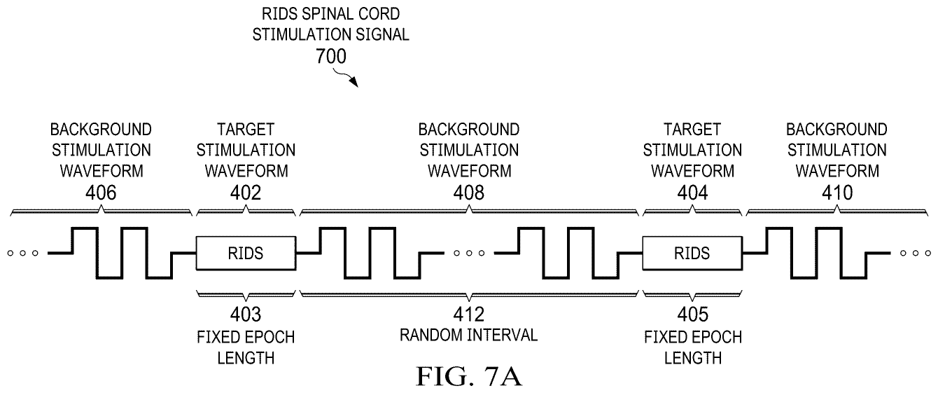

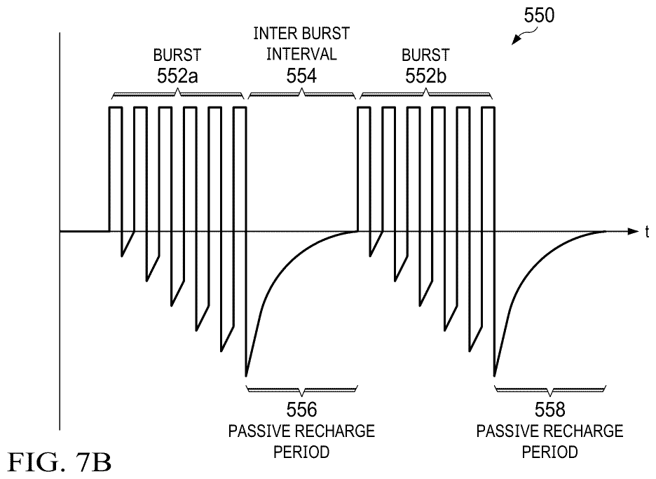

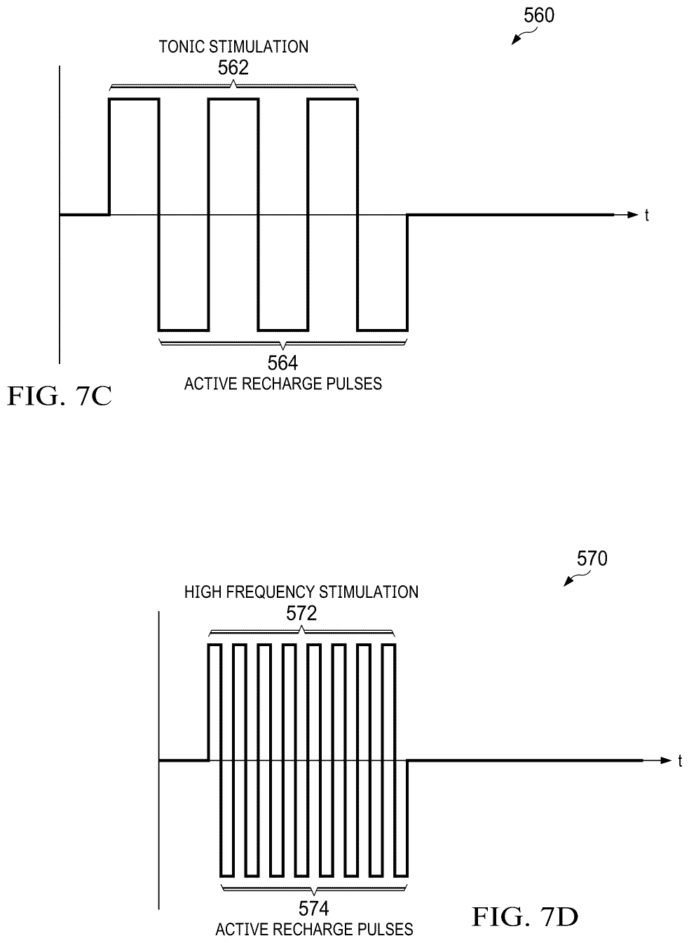

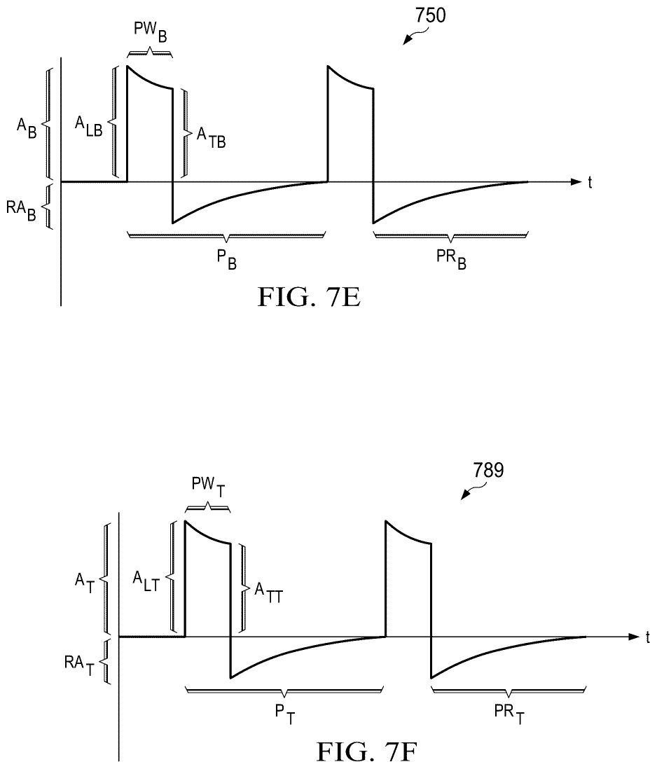

Referring to FIG. 7A, a preferred RIDS stimulation signal 700 will be further described.

RIDS stimulation signal 700 consists of a background stimulation waveform, such as background waveforms 406, 408 and 410 with target stimulation waveforms, such target stimulation waveforms 402 and 404 of a specified epoch length, such as fixed epoch lengths 403 and 405, separated by random intervals in time, such as random interval 412.

Each occurrence of the target waveform is referred to as an “epoch.” During the epoch, the target waveform is substituted for the background waveform. Each epoch lasts for a finite period of time referred to as an “epoch length.” The random interval is defined by a “target probability.” The target probability is the probability that the target waveform will be substituted for the background waveform.

The background waveform is defined by at least the parameters of amplitude, pulse width and frequency. The target waveform is defined by at least the parameters of amplitude, pulse width, frequency, epoch length, and target probability, as will be further described. Alternatively, the target waveform may be defined by the parameters of amplitude, pulse width, frequency, epoch length, and the number of epochs desired each day, as will be further described.

In other embodiments, either the background waveform or the target waveform can be the absence of stimulation, such as would occur when the amplitude or the pulse width parameters are set to “0.” In any case, there must be a difference between the background waveform and the target waveform that is perceptible by the patient. For example, the background waveform and the target waveform can both be paresthesia-based stimuli provided that there is sufficient difference in at least one parameter so that the target waveform can be identified as distinct from the background waveform. If only one type of parameter such as amplitude, pulse width or frequency, is varied between the two waveforms, then the difference in that parameter should be at least ±20%. However, if multiple types of parameters are varied, such as amplitude and pulse width, or pulse width and frequency, then the difference in those parameters should be at least 5%. In another example, the background waveform may be paresthesia based and the target waveform may be the absence of stimulation or a paresthesia-free stimulus such that the target waveform is imperceptible itself but the difference in the target waveform compared to the background waveform makes the target perceptible by the patient. At least one of the target waveform and the background waveform must be perceptible by the patient. Said another way, both the target waveform and the background waveform may not be imperceptible.

The epoch length must be non-zero and long enough for the target waveform to be recognized by the patient. The minimum epoch length is dependent on how unique the target waveform is with respect to the background waveform. Shorter epoch lengths may be used if larger differences between the target waveform and the background waveform exist, and vice versa. Excessively long epoch lengths are not preferred, as the cumulative amount of time that the target waveform is active over the period of each day must be less than the cumulative amount of time that the background waveform is active over the same period, otherwise, the target waveform is perceived as the background waveform by the patient. Preferred epoch lengths range from 1 to 16 seconds.

The “target probability” may be calculated according to the following formula.

$$Target probability \% = \frac{Epoch length ( seconds ) \times desired number of epochs per day}{86400 ( seconds per day )} \times 100 \%$$

The target probability must be below 50%. Target probabilities above 50% can lead to habituation and reduce the efficacy of the selective attention task. If the target probability is too low, particularly with a short epoch length, the likelihood that the target waveform will be perceived by the patient is reduced. Optimal selections of epoch length and target probability, in one embodiment, should provide between 1 epoch and 48 epochs per day.

In preferred embodiments, the background waveform is defined by parameters in the ranges shown in Table 1.

TABLE 1

| Parameter | Background Waveform | |
|---|---|---|
| Amplitude | 0-25 | mA |
| Pulse Width | 0-20 | ms |
| Frequency | 0-1200 | Hz |

In a preferred embodiment, the target waveform is defined by the ranges shown in Table 2.

TABLE 2

| Parameter | Target Waveform | |
|---|---|---|
| Amplitude | 0-25 | mA |
| Pulse Width | 0-20 | ms |
| Frequency | 0-1200 | Hz |
| Epoch Length | 0.001-3600 | s |

| | |
|---|---|
| Target Probability | 0-49% |

As shown in FIGS. 7B, 7C and 7D, examples of preferred background waveforms will be further described.

FIG. 7B shows background burst stimulation waveform 550. Burst stimulation waveform 550 includes burst pulses 552a and 552b, separated by inter-burst interval 554. Burst pulse 552a is followed by passive recharge 556. Burst pulse 552a is followed by passive recharge 558. During the inter-burst interval, a passive charge balance occurs that dissipates any charge imbalance that might occur across the electrodes. In this example, there is an increasing pulse amplitude during the burst. Six, 1 millisecond pulses are followed by an inter-burst frequency of 500 Hz, delivered with a frequency of 40 Hz in a passive recharging waveform which maintains charge balance across the electrical contacts.

Referring to FIG. 7C, background tonic stimulation waveform 560 is shown. Tonic stimulation waveform 560 includes tonic stimulation pulses 562, followed by active recharge pulses 564. Tonic stimulation is a traditional form of neurostimulation that produces regular stimuli, generally applied with a frequency of about 10 to 150 Hz and an amplitude of up to about 25 mA. In tonic stimulation, pulses are delivered at a consistent frequency, pulse width, and amplitude.

Referring to FIG. 7D, background high frequency stimulation waveform 570 is shown. High frequency stimulation waveform 570 includes high frequency stimulation pulses 572, followed by active recharge pulses 574. High frequency spinal cord stimulation operates at a frequency typically greater than 500 Hz. This form of stimulation typically involves a low energy impulse that is not sensed by the patient. A high frequency spinal cord stimulation waveform consists of a square or sinusoidal waveform with a frequency ranging up to about 10 kHz, with amplitudes in the range of up to about 25 mA.

Referring then to FIG. 7E, another preferred background waveform, background waveform 750, will be further described.

Background waveform 750 may generally be considered a “modified” square wave. Background waveform 750 includes leading edge amplitude A_{LB}, followed by trailing edge amplitude A_{TB}. Pulses reoccur in a period, “P_{B}” between the leading edge of each pulse. Each pulse in background waveform 750 includes a pulse width, “PW_{B},” followed by a passive recharge period, “PR_{B}.” The negative amplitude of the recharge period leading edge is “RA_{B}.”

Referring then to FIG. 7F, a preferred target waveform, target waveform 789, will be further described.

Target waveform 789 may also generally be considered a “modified” square wave. Target waveform 789 includes leading edge amplitude, “A_{LT},” followed by trailing edge amplitude, “A_{TT},” pulses reoccur in a period, “P_{T},” between the leading edge of each pulse. Each pulse in target waveform 789 includes a pulse width, “PW_{T},” followed by a passive recharge period, “PR_{T}.” The negative amplitude of the recharge period leading edge is “RA_{T}.”

Table 3 and Table 4 below show prophetic examples for a RIDS stimulation signal including a pairing of a background waveform with a target waveform, an epoch length, and a target probability to a complete RIDS stimulation signal. In this example, background waveform BPA is paired with target waveform TPA, background waveform BPB is paired with target waveform TPB, and background waveform BPC is paired with target waveform TPC, as shown below.

TABLE 3
Background Waveform Parameters

Background
Waveform

| Parameters | A_{LB} | | A_{TB} | | f_{B} | | PW_{B} | | P_{B} | | RA_{B} | PR_{B} | |
|---|---|---|---|---|---|---|---|---|---|---|---|---|---|
| BPA | 12.5 | mA | 10.7 | mA | 800 | Hz | 100 | μs | 1250 | μs | 3.6 mA | 1175 | μs |
| BPB | 20 | mA | 17.1 | mA | 1.2 | kHz | 50 | μs | 830 | μs | 5.7 mA | 780 | μs |
| BPC | 6 | mA | 5.1 | mA | 400 | Hz | 100 | μs | 2500 | μs | 1.7 mA | 2400 | μs |

TABLE 4
Corresponding Target Waveform Parameters

| Target | | | | | | | | | |
|---|---|---|---|---|---|---|---|---|---|
| Waveform | | | | | | | | Epoch | Target |
| Parameters | A_{LT} | A_{TT} | f_{T} | PW_{T} | P_{T} | RA_{T} | PR_{T} | Length | Probability |
| TPA | 12.6 mA | 10.8 mA | 400 Hz | 120 μs | 2500 μs | 3.3 mA | 2100 μs | 8 s | 2% |
| TPB | 19.5 mA | 16.7 mA | 200 Hz | 200 μs | 1000 μs | 5.6 mA | 950 μs | 1 s | 3% |
| TPC | 6.1 mA | 5.2 mA | 90 Hz | 250 μs | 1250 μs | 1.74 mA | 1150 μs | 4 s | 1% |

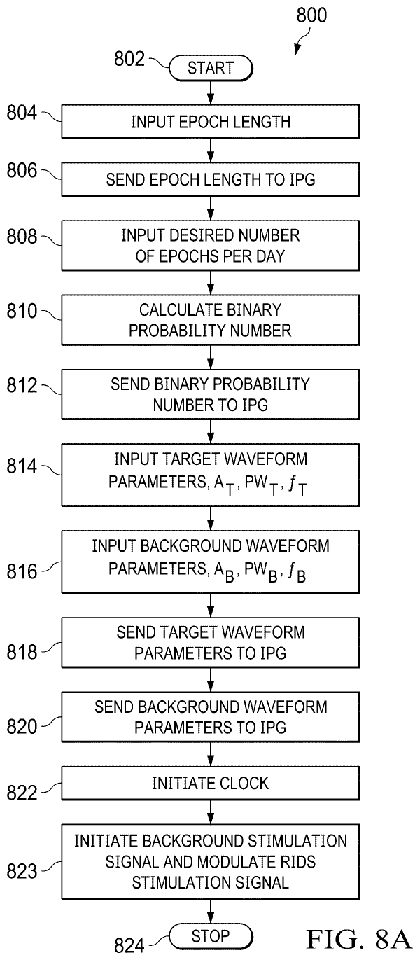

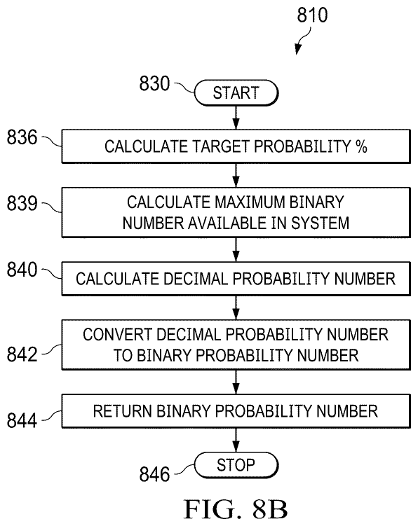

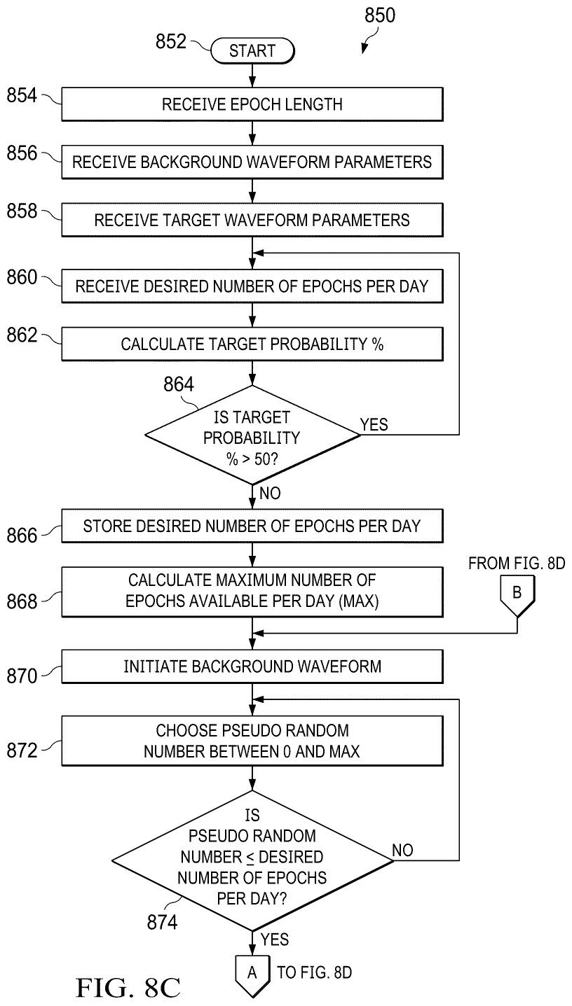

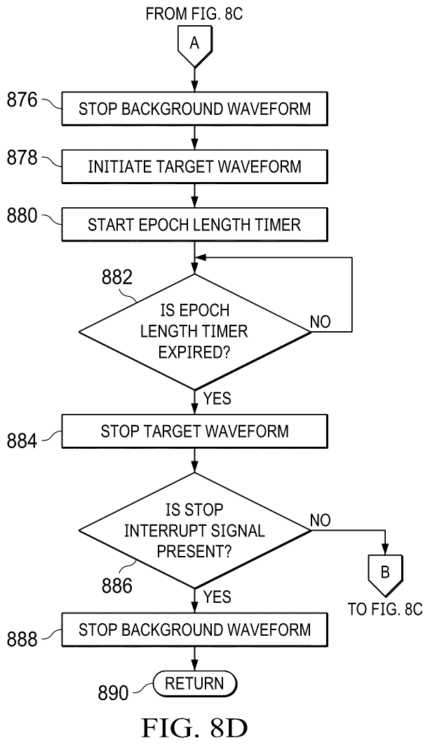

Referring to FIGS. 8A, 6B and 6C, preferred method 800 of operation of controller 520, in cooperation with controller 505, to deliver a RIDS stimulation signal, will be further described.

In this preferred embodiment, RIDS circuit 765 is present in controller 505 and operates automatically, after initialized, to modulate the RIDS stimulation signal, given input received from controller 520. All transfer of data between controller 520 and controller 505 is conducted by RF transceivers 706 and 771, and managed by CPUs 702 and 770, respectively. Method 800 takes the form of computer programs which are resident in memory 704 of controller 520 and memory 704 and memory 772 of controller 505. When activated, the system runs in a continuous cycle. Also in this example, the linear feedback shift register is a 16-bit register and the N−1 comparator is a 16-bit comparator. The epoch length is set to 16 seconds. The clock is set to generate 1 clock pulse every 16 seconds.

At step 802, the method begins.

At step 804, input keypad 718 of controller 520 is used to input an epoch length. Typical epoch lengths range from 1 to 16 seconds. Other epoch lengths are possible. Preferred choices for epoch length include 1, 2, 4, 8, and 16 seconds. Preferably, the epoch length is input as a decimal number which then is converted to a binary number before transmission to the IPG.

At step 806, the epoch length in binary form is sent to memory 772, which passes it to CPU 770, where it is used to set the clock frequency or clock rate.

At step 808, controller 520 receives an input from input keypad 708 of the desired number of epochs each day. Preferred numbers of epochs range between about 48 per day (averaging about two per hour) and 1 per day (about one every 24 hours). Other numbers of epochs are possible.

At step 810, the binary probability number is calculated, as will be further described. The binary probability number represents the target probability previously described.

At step 812, the binary probability number is sent to memory 784. Memory 784 sends the binary probability number to linear feedback shift register 781 where it is used to seed the register for initial operation. Memory 784 also loads the binary probability number to N−1 comparator 783, as input “B.”

At step 814, the target waveform parameters of amplitude, A_{T}, pulse width, PW_{T}, and frequency, f_{T}, are received from input keypad 708.

At step 816, background waveform parameters of amplitude, A_{B}, pulse width, PW_{B}, and frequency, f_{B}, are received from input keypad 708.

At step 818, the target waveform parameters are sent to memory 772.

At step 820, the background waveform parameters are sent to memory 772.

The target waveform parameters and the background waveform parameters are used by CPU 770 to set pulse modulator 762 and pulse generator 760 to produce the stimulation signal, which is sent to surgical lead 514.

At step 822, CPU 770 receives a “run” command from controller 520, and in response, sends a signal to clock 780, which initiates the clock to begin producing pulses at the clock rate. The clock is set to produce 1 pulse for each epoch length. At each clock pulse, the linear feedback shift register provides a new pseudo-random number, “A” and sends it to the N−1 comparator.

At step 823, the background stimulation signal is initiated and the RIDS stimulation signal is modulated as follows.

The A<B output of I_{C4} is normally low. If the A<B output of I_{C4} goes high, then N−1 comparator 783 has determined that the fifteen least significant bits of pseudo-random number “A” are less than the sixteen bits of probability number “B.” N−1 comparator 783 sends an edge triggered interrupt signal to the CPU, which in response, retrieves the parameters which define the target waveform from memory and sends them to pulse modulator 762. Pulse modulator 762 modulates the signal and sends it to pulse generator 760, which generates the target signal and sends it to the surgical lead. At the next clock cycle, the CPU inverts the edge triggered interrupt, retrieves the parameters which define the background waveform and resends them to the pulse modulator. The pulse modulator then modulates the background signal and sends it to the pulse generator which generates the background waveform stimulation signal and sends it to the surgical lead. The cycle repeats when the CPU receives the next edge triggered interrupt from the N−1 comparator.

At step 824, the method concludes.

Referring to FIG. 8B, calculating the binary probability number of step 810 will be further described. Preferably this method is executed as a program resident in memory in controller 520.

At step 830, the method begins.

At step 836, the target probability percentage, in decimal form, is calculated according to the following equation:

$$Target probability \% = \frac{Epoch length ( seconds ) \times Desired number of epochs per day}{86400 ( seconds per day )} \times 100$$

At step 839, the maximum binary number available in the system is calculated according to the following equation:

$$Maximum binary number available = {2}^{N}$$

### Where:

N=number of bits in the linear feedback shift register and the N−1 comparator.

At step 840, the decimal probability number is calculated according to the following equation:

$$Decimal probability number = target probability \% \times maximum binary number available$$

At step 842, the decimal probability number is converted to an unsigned binary number, the “binary probability number.” In a preferred embodiment, the binary probability number is an unsigned 16-bit number.

At step 844, the binary probability number is returned.

At step 846, the method concludes.

The following calculations provide preferred examples of step 810.

### Example 1—Given:

$$N = 16 bit registers for the linear \\ feedback shift register and the ( N - 1 ) comparator \\ Epoch length = 16 seconds \\ Desired number of epochs per day = 1 \\ Target probability \% = \frac{16 ( seconds ) \times 1 ( per day )}{86400 ( seconds per day )} \times 100 = 0.185 \% \\ Maximum binary number available = {2}^{N} = {2}^{16} = 65535 \\ Decimal probability number = 0.185 \times 65533 = 1212 \\ 16 - bit binary probability number = 0000010010111100$$

### Example 2—Given:

$$N = 8 bit registers for the linear \\ feedback shift register and the ( N - 1 ) comparator \\ Epoch length = 4 seconds \\ Desired number of epochs per day = 48 \\ Target probability \% = \frac{4 ( sesconds ) \times 48 ( per day )}{86400 ( seconds per day )} \times 100 = 0.222 \% \\ Maximum binary number available = {2}^{N} = {2}^{8} = 256 \\ Decimal probability number = 0.222 \times 256 = 57 \\ 8 - bit binary probability number = 00111001$$

Referring to FIGS. 8C and 8D, an alternate preferred method of operation of controller 520, in cooperation with controller 505, to deliver a RIDS stimulation signal will be further described.

In this preferred embodiment, method 850 takes the form of a computer program which is resident in memory 772 of controller 505. When activated, the program runs in a continuous cycle. In this embodiment, RIDS circuit 765 is not necessarily present in the controller (or is unused), and the RIDS stimulation signal waveforms are generated by software. In an alternate embodiment, the computer program may be initiated in the unlikely event that a fault develops in RIDS circuit 765, so that the IPG may continue to operate without the need for surgical replacement.

At step 852, the method begins.

At step 854, the epoch length is received from controller 520, in binary form, as previously described. In a preferred embodiment, a message is displayed on display 710 requesting an input of epoch length, in seconds. If received in units of seconds, the controller converts the input into binary form at this step.

At step 856, the background waveform parameters of amplitude, pulse width, and frequency are received from controller 520.

At step 858, the target waveform parameters of amplitude, pulse width, and frequency are received from controller 520.

At step 860, the desired number of epochs per day is received from controller 520. In a preferred embodiment, a message is also displayed on display 710 requesting an input of the desired number of epochs per day from the user.

At step 862, the target probability percentage is calculated according to the following equation:

$$Target probability \% = \frac{Epoch length ( in seconds ) \times desired number of epochs ( per day )}{86400 ( seconds per day )} \times 100$$

As step 864, CPU 770 determines whether or not the target probability percentage is equal to or greater than 50%. If so, the CPU returns to 860. If not, the CPU moves to step 866.

At step 866, the CPU stores the desired number of epochs per day.

At step 868, the CPU calculates the maximum number of epochs possible per day, “MAX,” according to the following equation:

$$Maximum number of epochs per day ( MAX ) = \frac{86400}{Epoch length}$$

At step 870, the CPU initiates the background waveform by reading the background waveform parameters from memory and sending a signal to the waveform modulator, which modulates the waveform and sends it to the waveform generator. The waveform generator creates the stimulation signal and sends it to the surgical lead, as previously described.

At step 872, the CPU generates a pseudo-random number between 0 and MAX.

At step 874, the CPU determines whether or not the pseudo random number is less than or equal to the desired number of epochs per day. If not, the CPU returns to step 872. If so, the CPU moves to step 876.

At step 876, the CPU stops the background waveform.

At step 878, the CPU initiates the target waveform, as previously described.

At step 880, the CPU starts the epoch length timer. In a preferred embodiment, the epoch length timer runs for exactly one epoch length.

At step 882, the CPU determines whether or not the epoch timer has expired. If not, the CPU loops until the epoch length timer expires. If so, the CPU moves to step 884.

At step 884, the CPU stops the target waveform.

At step 886, the CPU checks for a “stop” interrupt signal from controller 520. If no stop interrupt signal is present, the CPU returns to step 870. If a stop interrupt signal is present, CPU moves to step 888.

At step 888, the CPU stops the background waveform.

At step 890, the CPU returns.

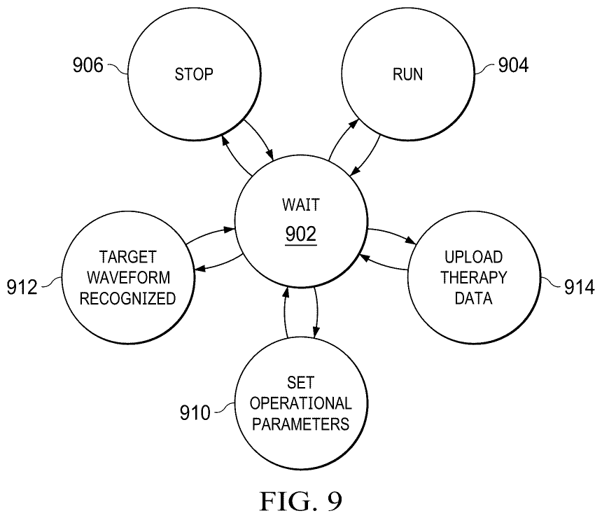

Referring to FIG. 9, the preferred states of controller 520 will be further described.

At state 902, CPU 702 enters a waiting posture and continually polls input keypad 708.

At state 904, upon receipt of a “run” signal from the input device, CPU 702 transmits a “run” signal to RF transceiver 706. The RF transceiver then transmits the “run” signal to RF transceiver 771 for transmission to the IPG. After transmission, CPU 702 returns to wait state 902.

At state 906, if a “stop” signal is received from input keypad 708, CPU 702 passes a “stop” signal to RF transceiver 706 which in turn sends the “stop” signal to RF transceiver 771 for transmission to the IPG. CPU 702 then returns to wait state 902.

At state 910, if a “set operational parameters” signal is received from input keypad 708, CPU 702 displays an information request on display 710 and then waits for data input from input keypad 708. CPU 702 then receives a set of target waveform parameters, epoch length (in seconds), a target percentage, or alternatively a desired number of epochs per day (integer), (amplitude (mA)), pulse width (s), and frequency (Hz) and a set of background waveform parameters (amplitude (mA), pulse width (s) and frequency (Hz)) from input keypad 708. These parameters are stored in memory 704 where they are uploaded to RF transceiver 706 for transmission to the IPG. CPU 702 then returns to wait state 902.

At state 912, if a “target waveform recognized” signal is received from input keypad 708, CPU 702 displays an acknowledgement on display 710. During therapy, the “target waveform recognized” button press is received when the patient recognizes the target waveform as being different than the background waveform. The time and date of the occurrence of the target waveform, as well as the date and time of the button press are stored in a running table in memory.

An prophetic example of a running table is shown below.

TABLE 5

| | Target Waveform |
|---|---|
| Target Signal Occurrence | Recognized Button Press |

| Date | Time | Date | Time |
|---|---|---|---|
| Mar. 13, 2024 | 5:01 | Mar. 13, 2024 | 5:02 |
| Mar. 14, 2024 | 16:34 | Mar. 14, 2024 | 16:34 |
| Mar. 15, 2024 | 2:37 | Mar. 15, 2024 | 2:38 |
| . | . | . | . |
| . | . | . | . |
| . | . | . | . |
| Apr. 1, 2024 | 11:45 | XX | XX |
| Apr. 2, 2024 | 23:20 | Apr. 2, 2024 | 23:21 |
| Apr. 3, 2024 | 6:07 | Apr. 3, 2024 | 6:07 |

As can be seen from Table 5, entries for a date and time for the “target waveform recognized” button press are missing for Apr. 1, 2024, indicating that the target signal was not recognized by the patient when it occurred.

At state 914, if an “upload therapy data” signal is received from input keypad 708, then CPU 702 moves the running table from memory to RF transceiver 706 where it is transmitted to administrator device 540.

In all cases, an appropriate message is sent to display 710, from CPU 702, to continuously display the operational state of controller 520 and any required information or instructions for the user.

In a preferred embodiment, CPU 712 of the administrator device is programed to continually poll RF transceiver 716, and immediately receives the running table, when transmitted, and automatically stores it in memory 714, for later retrieval and display based on instructions from input keypad 718. The running table is used to evaluate whether or not each epoch is consistently recognized above the background waveform by the patient, as is required for effective therapy. A percentage of the time that the target waveform epoch is recognized may be calculated according to the following formula:

$$Recognition rate = \frac{Number of “ target waveform recognized ” button pressses}{Total number of target waveform occurances} \times 100 \%$$

Preferably, each should be recognized by the patient when it occurs, or a recognition rate of about 100% of the time that the RIDS stimulation signal is active. However, recognition rates above 20% are also effective.

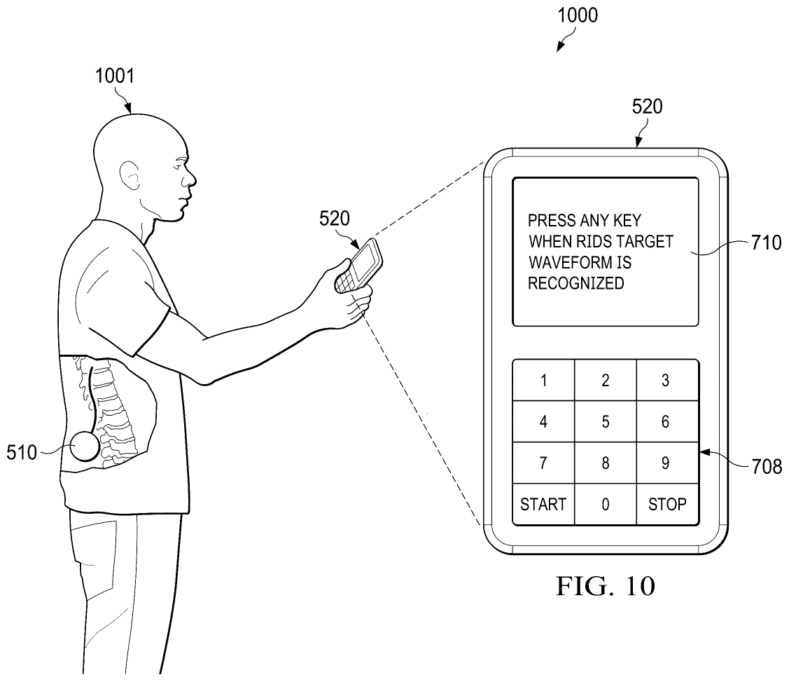

Referring then to FIG. 10, a preferred therapy setup will be further described.

Therapy setup 1000 includes patient 1001 in which is implanted IPG 510 and the surgical lead, as previously described. Controller 520 is in communication with the IPG and receives signals from it and sends control signals to it. Display 710 displays messages for patient 1001 and receives physical button presses on input keypad 708, as previously described.

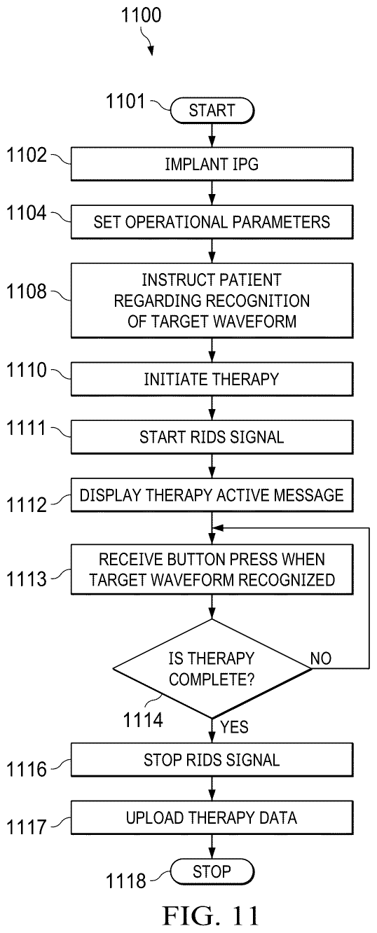

Referring then to FIG. 11, a preferred method of therapy 1100 using a preferred RIDS stimulation signal will be further described.

At step 1101, the method begins.

At step 1102, an IPG and surgical lead are implanted, as previously described.

At step 1104, the IPG is programmed with a set of RIDS signal operational parameters, as previously described.

Importantly, at step 1108, the patient is instructed regarding the RIDS therapy. In one preferred embodiment, the patient is instructed to complete a physical task, such as pressing a button on input keypad 708 of controller 520, every time that the target waveform is perceived or recognized by the patient over the background waveform. In another preferred embodiment, the patient is instructed to imagine undertaking a specific physical task, such as pressing a button, when the target waveform is perceived or recognized by the patient over the background waveform.

At step 1110, therapy is initiated. Preferably, therapy is initiated by pressing the “run” button on controller 520. At step 1111, controller 520 sends a signal to controller 505, of IPG 510 which activates the RIDS stimulation waveform, including the randomly dispersed target waveform and the background waveform, as previously described.

At step 1112, controller 520 displays a “therapy active” (or equivalent) message on display 710.

At step 1113, the patient presses an input button on controller 520 when the target waveform is recognized. The date and time at which the target waveform was transmitted to the surgical lead and the date and time that the button is pressed are stored in a running table in memory of controller 505. If no button press signal is received when the target waveform is transmitted to the surgical lead, then only the date and time of the transmission of the target waveform to the surgical lead are stored.

At step 1114, a clinical decision is made as to whether or not therapy is complete. If not, the method returns to step 1112. If so, the method moves to step 1116.

At step 1116, the RIDS signal is stopped by entering a “stop” command on controller 520. Controller 520 then sends a signal to controller 505 of IPG 510, which stops the RIDS signal.

At step 1117, an “upload therapy data” command is received from controller 520, and the running table is sent to it for analysis.

At step 1118, the therapy session concludes.

The following prophetic examples of patient case studies of RIDS signal therapy implementations are provided.

Patient Name: John Doe 1

Age: 42

Occupation: Construction worker

Medical History, Previous Medical Conditions: No significant medical history prior to the injury.

Allergies: None known.

Injury Details, Date of Injury: Jun. 15, 2023

Mechanism of Injury: Fall from a height at a construction site, resulting in a severe lumbar spine injury.

Initial Treatment: Emergency decompression surgery followed by stabilization of the lumbar spine.

Postoperative Course: Despite successful surgery, John experienced persistent and debilitating pain that did not respond to conventional pain management strategies, including medications, physical therapy, and nerve blocks.

Primary Diagnosis: Failed Back Surgery Syndrome (FBSS)

Secondary Complications: Development of neuropathic pain, characterized by burning sensations and electrical-like shocks in the lower extremities.

Treatment Plan: After a thorough evaluation by a multidisciplinary team and failure of conservative treatments, it was decided to proceed with a trial of spinal cord stimulation.

Trial Period: A temporary spinal cord stimulator was implanted for a week to assess the efficacy of pain relief.

Outcome of Trial: Significant reduction in pain scores by 50% and improvement in quality of life.

Definitive Treatment: Based on the successful trial, a permanent spinal cord stimulator was implanted and the RIDS therapy was instituted to reduce habituation.

Procedure Details: The procedure involved placing electrodes in the epidural space near the area of the injured spinal cord and implanting a pulse generator in the flank. The pulse generator was programmed with a RIDS stimulation signal employing a background waveform having an amplitude of 12.5 mA, a pulse width of 1250 μs, and a frequency of 800 Hz, and a target waveform having an amplitude of 12.6 mA, a pulse width of 75 μs and a frequency of 250 Hz. An epoch length of 4 s and a target probability of 0.5% were used.

Post-Implantation Care: John underwent a rehabilitation program to maximize the benefits of the stimulator and was educated on managing the device settings and the RIDS therapy. RIDS therapy was conducted two days a week for a 6-month period.

Follow-Up: At the 6-month follow-up, John reported sustained pain relief and was able to return to a modified form of work and engage more actively in daily activities. The RIDS running table indicated about a 95% recognition rate, indicating that the target signal was sufficiently different from the background signal to be easily recognized.

Conclusion: The spinal cord stimulator implant and RIDS therapy significantly improved John's pain management and overall quality of life, demonstrating its value as a treatment option for FBSS when other therapies have failed.

### Example 2

Patient Name: Jane Smith

Age: 55

Occupation: Software Developer

Medical History, Previous Medical Conditions: Chronic lower back pain due to degenerative disc disease.

Allergies: None known.

Previous Interventions: Laminectomy and discectomy, followed by the implantation of a spinal cord stimulator two years prior.

Presenting Problem: Jane began to experience a gradual decrease in the efficacy of her spinal cord stimulator over the past six months, reporting only 20% pain relief compared to the initial 70% relief following implantation.

Assessment: A comprehensive evaluation was conducted, including a device interrogation, which confirmed proper function of the stimulator. No hardware issues were detected. Jane's pain management specialist diagnosed her with habituation to the spinal cord stimulator.

Intervention: After discussing various options, Jane agreed to a program of RIDS therapy where her spinal cord stimulator was reprogrammed with the RIDS stimulation signal and RIDS therapy was instituted for a period of 3-months to reduce the habituation effect. The stimulator was reprogrammed with a RID stimulation signal with a background waveform having an amplitude of 6 mA, a pulse width of 350 μs and a frequency of 400 Hz. The background waveform was paired with a target waveform having an amplitude of 6.1 mA, a pulse width of 100 μs, a frequency of 90 Hz, an epoch length of 2 s and a number of desired epochs per day of 12.

Outcome: Following the RIDS therapy, Jane reported an improvement in pain relief to 50%. At a follow-up three months later, Jane reported an improvement of pain relief of 75%. The RIDS running table indicated about a 90% recognition rate, indicating that the target signal was sufficiently different from the background signal to be easily recognized.

### Example 3

Patient Name: John Doe 2

Age: 45

Occupation: Construction Worker

Medical History: Chronic lower back pain following a work-related injury five years ago. Multiple conservative treatments attempted including physical therapy, medications, and epidural steroid injections with minimal relief.

Injury and Treatment Course: John sustained a lumbar spine injury resulting in persistent and debilitating neuropathic pain. Despite exhaustive conservative management, his pain remained severe, rated 8/10 on the Visual Analog Scale (VAS), significantly impairing his quality of life and ability to work.

A multidisciplinary team evaluated John and recommended a trial of spinal cord stimulation (SCS) using the RIDS therapy after he met the criteria for the therapy. The trial period showed promising results, with a 50% reduction in pain. Consequently, a permanent SCS device was implanted and RIDS therapy implemented. The stimulator was programmed with a RIDS stimulation signal, employing a background waveform having an amplitude of 20 mA, a pulse width of 50 μs and a frequency of 1.2 kHz. The background waveform was paired with a target waveform having an amplitude of 19.5 mA, a pulse width of 500 μs, and a frequency of 60 Hz. An epoch length of 1 second and a target probability of 0.1% were used.

Outcome: Within six months post-implantation, John reported a gradual decrease in his pain. After several follow-up appointments and adjustments to the SCS settings, the decision was made to continue RIDS therapy monthly to sustain efficacy and reduce habituation. The RIDS running table indicated about a 90% recognition rate indicating that the target signal was significantly different from the background signal to be recognized by the patient. John was then able to resume daily activities.

It will be appreciated by those skilled in the art that changes could be made to the embodiments described above without departing from the broad inventive concept thereof. It is understood, therefore, that this disclosure is not limited to the particular embodiments disclosed, but it is intended to cover modifications within the spirit and scope of the present disclosure as defined by the appended claims.

1. A spinal cord stimulator comprising:
a first processor;
an electrode adapted to send a background stimulation signal and a target stimulation signal;
the target stimulation signal being different than the background stimulation signal;
a pulse modulator, operatively connected to the first processor;
a pulse generator, operatively connected to the pulse modulator and the electrode; and
a first memory, operatively connected to the first processor, containing a first set of instructions that when executed cause the first processor to carry out the steps of:
delivering the background stimulation signal from the pulse generator to the electrode;
initiating an epoch timer to create a randomized trigger signal;
delivering the target stimulation signal from the pulse generator to the electrode, based on the randomized trigger signal; and
re-initiating the background stimulation signal after delivery of the target stimulation signal.

2. The spinal cord stimulator of claim 1, wherein the epoch timer further comprises:
a clock;
a linear feedback shift register, operatively connected to the clock;
the first memory, operatively connected to the clock and the linear feedback shift register; and
an N−1 comparator, operatively connected to the first memory and the linear feedback shift register.

3. The spinal cord stimulator of claim 2, wherein the linear feedback shift register further comprises:
an N bit shift register, having a register input and a register output; and
a set of logic gates, operatively connected to the N bit shift register.

4. The spinal cord stimulator of claim 3, wherein the set of logic gates further comprises:
a first XOR gate, having a first input and a second input and a first output, the first input tapped to the N bit shift register at a first bit location, and the second input tapped to the N bit shift register at a second bit location;
a second XOR gate, having a third input and a fourth input and a second output, the first input connected to the first output, the second input tapped to the N bit shift register at a third bit location; and
a third XOR gate, having a fifth input and a sixth input, and a third output, the fifth input connected to the second output, the sixth input tapped to the N bit shift register at a fourth bit location and the third input connected to the register input.

5. The spinal cord stimulator of claim 4, wherein the N−1 comparator further comprises:
an N bit comparator having a first set of inputs and a second set of inputs, and generating a comparator output;
the first set of inputs connected to the first memory;
the second set of inputs connected to the register output; and
the comparator output connected to the first processor.

6. The spinal cord stimulator of claim 5, wherein the first set of instructions further comprises instructions that when executed cause the first processor to carry out the steps of:
receiving an epoch length;
receiving a desired number of epochs for a time period;
calculating a binary probability number, having N bits;
receiving a set of target waveform parameters;
receiving a set of background waveform parameters;
activating the clock to produce a pulse based on the epoch length;
initiating the background stimulation signal; and
modulating the target stimulation signal based on the pulse and the binary probability number.

7. The spinal cord stimulator of claim 6, wherein the step of modulating further comprises:
generating a pseudo-random number, having N bits, with the linear feedback shift register;
comparing N−1 bits of the pseudo-random number with the binary probability number in the N−1 comparator; and
if the N−1 bits of the pseudo-random number are less than the N bits of the binary probability number, then sending an interrupt signal to the first processor from the N−1 comparator.

8. The spinal cord stimulator of claim 7, wherein the first set of instructions further comprises instructions that when executed, cause the first processor to carry out the steps of:
upon receipt of the interrupt signal;
retrieving the set of target waveform parameters from the first memory;
sending the set of target waveform parameters to the pulse modulator to create a modulated target signal;
sending the modulated target signal to the pulse generator; and
generating the target stimulation signal based on the modulated target signal.

9. The spinal cord stimulator of claim 8, wherein the first set of instructions further comprises instructions that when executed cause the first processor to carry out the steps of:
maintaining the target stimulation signal for the epoch length.

10. The spinal cord stimulator of claim 9, wherein N is one of a group of 4, 8, 16, 32 and 64.

11. The spinal cord stimulator of claim 6, wherein the set of target waveform parameters includes an amplitude, a pulse width, a frequency, the epoch length, and a target probability related to the binary probability number.

12. The spinal cord stimulator of claim 11, wherein the target probability is between about 0% and about 49%.

13. The spinal cord stimulator of claim 11, wherein the epoch length is between about 0.001 seconds and about 3,600 seconds.

14. The spinal cord stimulator of claim 13, wherein the epoch length is one of a group of 1 second, 2 seconds, 4 seconds, 8 seconds, and 16 seconds.

15. The spinal cord stimulator of claim 11, wherein the amplitude is between about 0 mA and about 25 mA.

16. The spinal cord stimulator of claim 11, wherein the pulse width is between about 0 μs and about 20 ms.

17. The spinal cord stimulator of claim 11, wherein the frequency is between about 0 Hz and about 1,200 Hz.

18. The spinal cord stimulator of claim 11, wherein the epoch length and the target probability provide between 1 and 48 epochs per day.

19. The spinal cord stimulator of claim 6, wherein the set of background waveform parameters includes an amplitude, a pulse width, and a frequency.

20. The spinal cord stimulator of claim 18, wherein the amplitude is between about 0 mA and about 25 mA.

21. The spinal cord stimulator of claim 18, wherein the pulse width is between about 0 μs and about 20 ms.

22. The spinal cord stimulator of claim 18, wherein the frequency is between about 0 Hz and about 1,200 Hz.

23. The spinal cord stimulator of claim 1, wherein the first set of instructions further comprises instructions that when executed cause the first processor to carry out the steps of:
receiving an epoch length;
receiving a set of background waveform parameters;
receiving a set of target waveform parameters;
receiving a desired number of epochs for a time period;
deriving a maximum number of epochs for the time period;
after the step of delivering the background stimulation signal, choosing a pseudo-random number between zero and the maximum number of epochs for the time period; and
if the pseudo-random number is less than or equal to the desired number of epochs for the time period, then deactivating the background stimulation signal and delivering the target stimulation signal for the epoch length.

24. The spinal cord stimulator of claim 23, wherein the first set of instructions further comprises instructions that when executed cause the first processor to carry out the steps of:
deriving a target probability percentage based on the epoch length and the desired number of epochs for the time period; and
if the target probability percentage is greater than 50%, then returning to the step of receiving the desired number of epochs for the time period.

25. The spinal cord stimulator of claim 23, further comprising:
a second processor, in operative communication with the first processor; and
an input device, operatively connected to the second processor and a second memory, the second memory containing a second set of instructions that when executed cause the second processor to carry out the steps of:
receiving an indicator signal, from the input device, indicating a perception of the target stimulation signal by a patient; and
storing the indicator signal in the second memory.

26. The spinal cord stimulator of claim 25, further comprising:
a third processor, in communication with the second processor; and
a third memory, operatively connected to the third processor, containing a third set of instructions that when executed caused the third processor to carry out the step of:
receiving the indicator signal from the second processor.

27. A method of spinal cord stimulation comprising the steps of:
providing an implanted pulse generator, operatively connected to an implanted electrode;
sending a background stimulation signal from the implanted pulse generator to the implanted electrode; and
substituting a target stimulation signal, different than the background stimulation signal, for the background stimulation signal, at a random time interval.

28. The method of claim 27, further comprising:
observing a recognition of a target stimulus perception elicited from a patient upon a patient's perception of the target stimulation signal.

29. The method of claim 28, further comprising:
providing a set of instructions to the patient to execute an imaginary task upon the recognition.

30. The method of claim 28, further comprising:
providing a set of instructions to the patient to execute a physical task upon the recognition.

31. The method of claim 28, further comprising:
providing a processor, operatively connected to the implanted pulse generator;
providing an input device, operatively connected to the processor; and
receiving a signal, from the input device, indicating the recognition.

32. A method of spinal cord stimulation comprising the steps of:
providing a processor;
providing an implanted pulse generator, operatively connected to the processor;
providing an implanted electrode, operatively connected to the implanted pulse generator;
sending a background stimulation signal from the implanted pulse generator to the implanted electrode;
calculating, at the processor, a random time interval; and
sending a target stimulation signal, from the implanted pulse generator, to the implanted electrode, based on the random time interval.

33. The method of claim 32, further comprising:
observing a recognition of a target stimulus perception elicited from a patient upon a patient's perception of the target stimulation signal.

34. The method of claim 33, further comprising:
providing a set of instructions to the patient to execute a virtual task upon the recognition.

35. The method of claim 33, further comprising:
providing a set of instructions to the patient to execute a physical task upon the recognition.
